(19) United States
(12) Patent Application Publication
(10) Pub. No.: US 2018/0243517 A1
(43) Pub. Date: Aug. 30, 2018

(54) Elbow Braces for Intravenous Therapy and Related Methods

(71) Applicant: Cynthia Edwards, Bronx, NY (US)

(72) Inventor: Cynthia Edwards, Bronx, NY (US)

(21) Appl. No.: 15/445,432

(22) Filed: Feb. 28, 2017

(51) Int. Cl. A61M 5/52

(52) U.S. Cl. CPC A61M 5/52; A61M 2209/088

(57) ABSTRACT

This disclosure includes elbow braces for intravenous therapy and related methods. Some braces have a rigid frame, wherein the frame includes: a width and a length longer than the width; a first end having a first surface configured to contact an arm of a patient and a second end spaced along the length from the first end and having a second surface configured to contact the arm, wherein the second surface is configured to be disposed at an angle relative to the first surface that is between approximately 10 degrees and 45 degrees; and an opening extending through the frame; and a cushion configured to be disposed onto at least a portion of a peripheral edge of the frame.

### BACKGROUND

### 1. Field of Invention

The present invention relates generally to braces, and more specifically, but not by way of limitation, to elbow braces for intravenous therapy and related methods.

### 2. Description of Related Art

Examples of braces for intravenous therapy are disclosed in U.S. Pat. Nos. (1) 3,722,508; and (2), 6,526,981, and U.S. Patent Pub. Nos.: (1) 2013/0012883; and (2) 2014/0060547.

### SUMMARY

Some embodiments of the present elbow braces comprise a rigid frame, wherein the frame includes: a width and a length longer than the width; a first end having a first surface configured to contact an arm of a patient and a second end spaced along the length from the first end and having a second surface configured to contact the arm, wherein the second surface is configured to be disposed at an angle relative to the first surface that is between approximately 10 degrees and 45 degrees; and an opening extending through the frame; and a cushion configured to be disposed onto at least a portion of a peripheral edge of the frame.

In some embodiments, the opening includes a length and a width, wherein the length is longer than the width; and the length of the opening is at least half of the length of the frame. In some embodiments, the opening is sized such that, when the frame is disposed on the arm of the patient, an intravenous therapy site on the arm is visible through the opening.

In some embodiments, the first surface and the second surface are configured to contact an anterior side of the arm. In some embodiments, the first surface and the second surface are configured to contact a posterior side of the arm.

In some embodiments, the width has a substantially uniform maximum dimension between the first end and the second end of the frame.

In some embodiments, the peripheral edge is raised relative to a portion of the frame such that, when the frame is disposed on the arm of the patient, the peripheral edge extends away from the arm.

Some embodiments of the present elbow braces include a rigid frame, wherein the frame includes: a width and a length longer than the width, wherein the width has a substantially uniform maximum dimension between a first end of the frame and a second end of the frame; an opening extending through the frame; and a cushion covering configured to be disposed onto at least a portion of a peripheral edge of the frame.

In some embodiments, the frame comprises a thermoplastic material.

In some embodiments, the first end of the frame is spaced along the length of the frame from the second end of the frame, and wherein the first end includes a first surface configured to contact an arm of a patient and the second end includes a second surface configured to contact the arm, wherein the second surface is configured to be disposed at an angle relative to the first surface that is between approximately 10 degrees and 45 degrees.

In some embodiments, the first surface and the second surface are configured to contact an anterior side of the patient's arm. In some embodiments, the first surface and the second surface are configured to contact a posterior side of the patient's arm.

In some embodiments, the opening is sized such that, when the frame is disposed on the arm of the patient, an intravenous therapy site is visible through the opening.

In some embodiments, the peripheral edge is raised relative to a portion of the frame such that, when the frame is disposed on the arm of the patient, the peripheral edge extends away from the patient's arm.

Some embodiments of the present elbow braces include one or more straps, each of which comprise a first end and a second end, wherein, when the frame is disposed on the arm of the patient, at least one of the one or more support straps is configured to wrap around the arm of the patient such that the first end and the second end of the strap are coupled to the frame.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the term “substantially” may be substituted with “within [a percentage] of” what is specified, where the percentage includes 0.1, 1, 5, and 10 percent.

The phrase “and/or” means and or. To illustrate, A, B, and/or C includes: A alone, B alone, C alone, a combination of A and B, a combination of A and C, a combination of B and C, or a combination of A, B, and C. In other words, “and/or” operates as an inclusive or.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus that “comprises,” “has,” or “includes” one or more elements possesses those one or more elements, but is not limited to possessing only those elements. Likewise, a method that “comprises,” “has,” or “includes,” one or more steps possesses those one or more steps, but is not limited to possessing only those one or more steps.

Any embodiment of any of the apparatuses, systems, and methods can consist of or consist essentially of—rather than comprise/have/include—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Further, a device or system that is configured in a certain way is configured in at least that way, but it can also be configured in other ways than those specifically described.

Some details associated with the embodiments are described above, and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1 is a perspective view of a first embodiment of the present elbow braces.

FIGS. 2 and 3 are top and bottom views, respectively, of the elbow brace of FIG. 1.

FIG. 4 is a cross-sectional side view of the elbow brace of FIG. 1, taken along line 4-4 of FIG. 3.

FIG. 5 is a perspective view of the elbow brace of FIG. 1, shown disposed on a patient's arm.

FIG. 6 is a perspective view of a second embodiment of the present elbow braces.

FIGS. 7 and 8 are top and bottom views, respectively, of the elbow brace of FIG. 6.

FIG. 9 is a cross-sectional side view of the elbow brace of FIG. 6, taken along line 9-9 of FIG. 7.

FIG. 10 is a perspective view of the elbow brace of FIG. 6, shown disposed on a patient's arm.

FIG. 11 is a perspective view of a third embodiment of the present elbow braces.

FIGS. 12 and 13 are top and bottom views, respectively, of the elbow brace of FIG. 11.

FIGS. 14 and 15 are first and second side views, respectively, of the elbow brace of FIG. 11.

### DETAILED DESCRIPTION

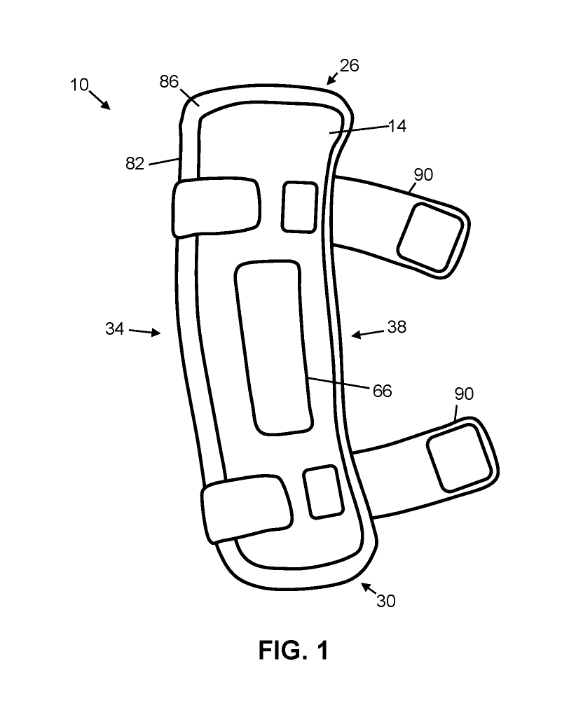

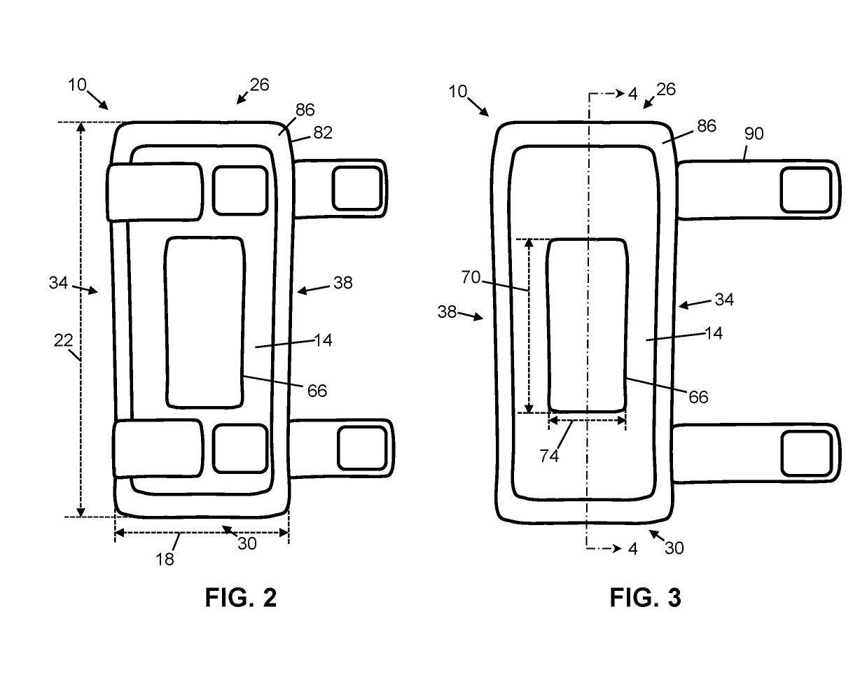

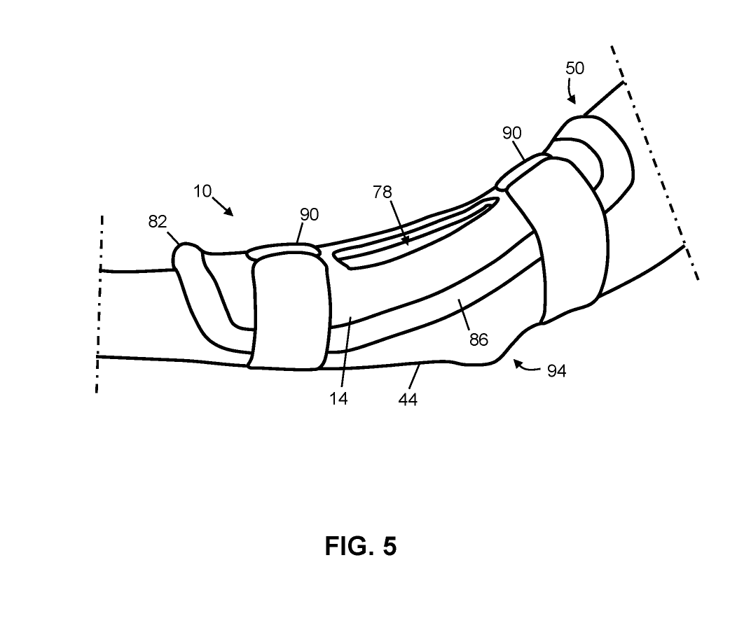

FIGS. 1-5 depict a first embodiment 10 of the present elbow braces. As described in more detail below, brace 10 is configured to immobilize an arm such that a medical procedure can be performed on the arm. Brace 10 can be used to immobilize an arm for a period of time (e.g., one or more hours, such as, for example, one, two, three, four or more hours), while maintaining the comfort of the patent. For example, by immobilizing at least a portion of an arm (e.g., 44), such as, for example, an elbow, in a position between approximately 10 degrees and 45 degrees of flexion, elbow pain is reduced (e.g., as compared to immobilizing the arm in an extended or hyperextended position). For further example, brace 10 can include a cushion (e.g., 86) to provided additional support, comfort, and/or protection to an arm (e.g., 44).

As shown, brace 10 includes a frame 14. For example, frame 14 may be rigid such that, when the frame is disposed on an arm 44 of a patient, the arm is immobilized at least in a direction of flexion and/or in a direction of extension. By immobilizing arm 44, frame 14 prevents movement of the arm that might otherwise disrupt a medical procedure being performed on the arm. For example, as described in further detail below, brace 10 may be suitable for use in intravenous (IV) therapy. During IV therapy, frame 14 may prevent undesired disconnection of a catheter (e.g., a hypodermic needle) and/or an IV tubing from arm 44 and may provide a visual of an IV therapy site such that a medical professional can visually assess whether the catheter and/or tubing is in a proper position relative to the arm.

Frame 14 may comprise any suitable material, such as, for example, a polymer (e.g., a thermoplastic material configured to immobilize, protect, and support injuries such as fractures, sprains, and/or strains), metal (e.g., stainless steel, aluminum, titanium, and/or the like), and/or the like.

Frame 14 includes a width 18 and a length 22, wherein the length is longer than the width. In this embodiment, frame 14 includes a first end 26 and a second end 30 that is spaced along length 22 from the first end. As shown, width 18 has a substantially uniform maximum dimension between first end 26 and second end 30 of frame 14. In the depicted embodiment, length 22 has a substantially uniform maximum dimension between a first side 34 and a second side 38 of frame 14. Thus, frame 14 may be characterized as having a rectangular shape. In other embodiments, a frame (e.g., 14) may include a shape that is elliptical, otherwise rounded, square, otherwise polygonal, and/or the like.

In this embodiment, first end 26 of frame 14 includes a first inner surface 42 configured to contact arm 44 of a patient and second end 30 of the frame includes a second inner surface 46 configured to contact the arm. First end 26 and/or second end 30 of frame 14 (e.g., and/or at least a portion of the frame between the first end and the second end) may include a concave portion such that the first end and/or the second end of the frame generally correspond to the convex contours of arm 44 of a patient.

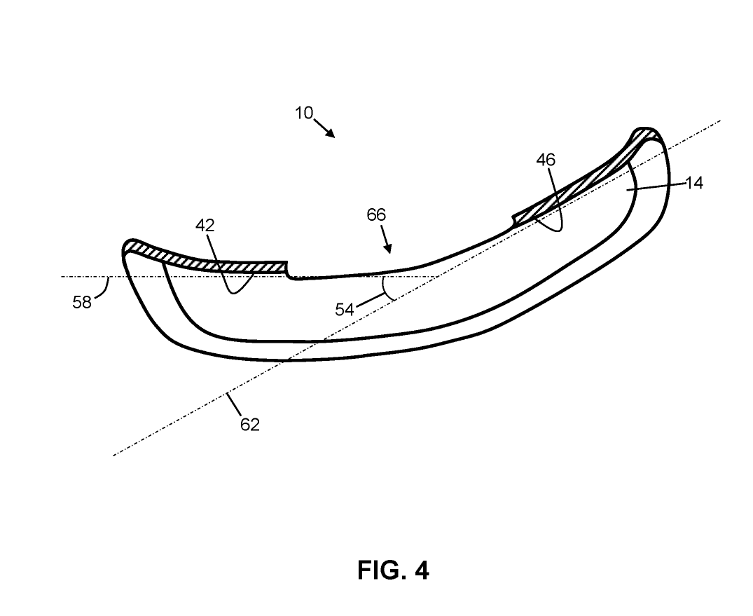

In the depicted embodiment, first inner surface 42 and/or second inner surface 46 of frame 14 may be configured to contact an anterior side 50 of arm 44. As shown, first inner surface 42 may be disposed at a non-zero angle relative to second inner surface. For example, second inner surface 46 may be disposed at a non-zero angle 54 relative to first inner surface 42 that is between approximately 10 degrees and 45 degrees (e.g., approximately any one of, or between any two, of the following: 10, 15, 20, 25, 30, 35, 40, and 45 degrees). Angle 54 may be measured between a first direction 58 that is parallel with at least a portion of first inner surface 42 and a second direction 62 that is parallel with at least a portion of second inner surface 46. As shown in FIG. 4, angle 54 is measured as the angle of arm 44 in a flexed position relative to the arm in an extended position. By immobilizing an arm (e.g., 44) in flexion (e.g., at a non-zero angle (e.g., 54) between a first inner surface (e.g., 42) and a second inner surface (e.g., 46), soreness, strain, and/or general discomfort of the arm may be reduced (e.g., as compared to immobilizing the arm in an extended or hyperextended position).

In the embodiment shown, brace 10 includes an opening 66 extending through frame 14. Opening 66 includes a length 70 and a width 74, where the length is longer than the width. For example, length 70 of opening 66 may be at least approximately 50 percent (e.g., approximately any one of, or between any two of, the following: 50, 55, 60, 65, 70, 75, 80, 85, and 90 percent) of length 22 of frame 14. Opening 66 may be sized such that, when frame 14 is disposed on arm 44 of a patient, an intravenous (IV) therapy site 78 on the arm is visible through the opening. For example, when an IV tubing and/or a catheter (e.g., a hypodermic needle) are coupled to the IV therapy site, opening 66 of brace 10 may provide a line of sight to the IV therapy site such that a medical professional can visually assess, without removing the brace, whether the IV tubing and/or the catheter are in a proper position relative to the arm.

In this embodiment, brace 10 can be configured such that an IV tubing and/or a catheter can extend through opening 66 to access IV therapy site 78 on arm 44. Brace 10 may (e.g., also) be configured such that an IV tubing can extend between frame 14 and arm 44 in order for a catheter to access IV therapy site 78 on the arm. At least by positioning the IV tubing between frame 14 and arm 44, brace 10 prevents undesired disconnection of the IV tubing and/or catheter from the arm if the arm inadvertently and/or purposely moves.

In the depicted embodiment, frame 14 includes a peripheral edge 82. As shown, at least a portion of edge 82 may be raised relative to another portion of frame 14 such that, when the frame is disposed on arm 44, the edge extends away from the arm. In this way and others, edge 82 of frame 14 avoids pinching skin of arm 44, for example, between the frame and one or more straps (e.g., 90) (described in further detail below).

Brace 10 may comprise a cushion 86. For example, cushion 86 may be configured to be disposed on at least a portion of edge 82 of frame 14 to protect arm 44 from lacerations, contusions, abrasions, and/or the like caused by contact with the edge. In this way and others, brace 10 provides additional comfort to a patient while the brace is disposed on arm 44. Cushion 86 may comprise any suitable material, such as, for example, a padded material (e.g., foam, felt, gauze, and/or the like), an inflated material (e.g., a material with one or more air-filled chambers), and/or the like.

Frame 14 may be secured to arm 44 in any suitable manner. For example, brace 10 includes one or more straps 90 configured to secure the brace to an arm 44 of a patient. For example, each strap 90 comprises a first end and a second end, wherein, when brace 10 is disposed on arm 44, at least one of the one or more support straps is configured to wrap around the arm such that the first end and the second end of the strap are both coupled to the frame. For example, a first end and/or a second end of each strap 90 may be coupled to frame 14 via a hook and loop fastener (e.g., Velcro), an adhesive, a button fastener, and/or the like. In other embodiments, a brace (e.g., 10) may include a sleeve configured to secure the brace to an arm (e.g., 44) by sliding onto the arm.

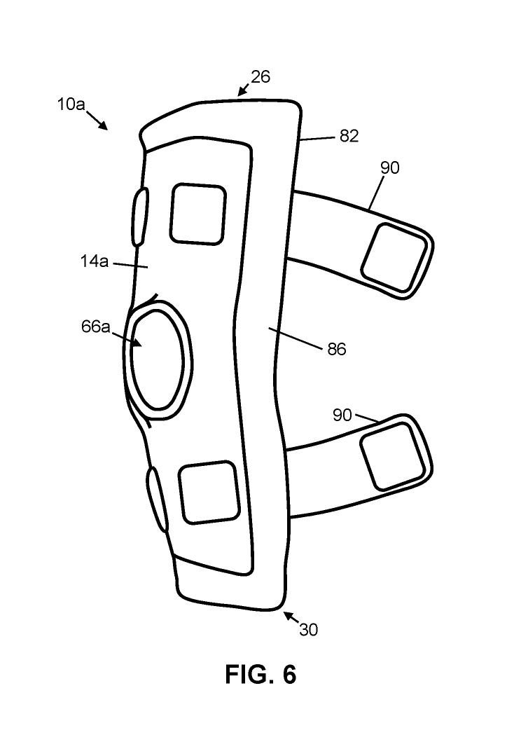

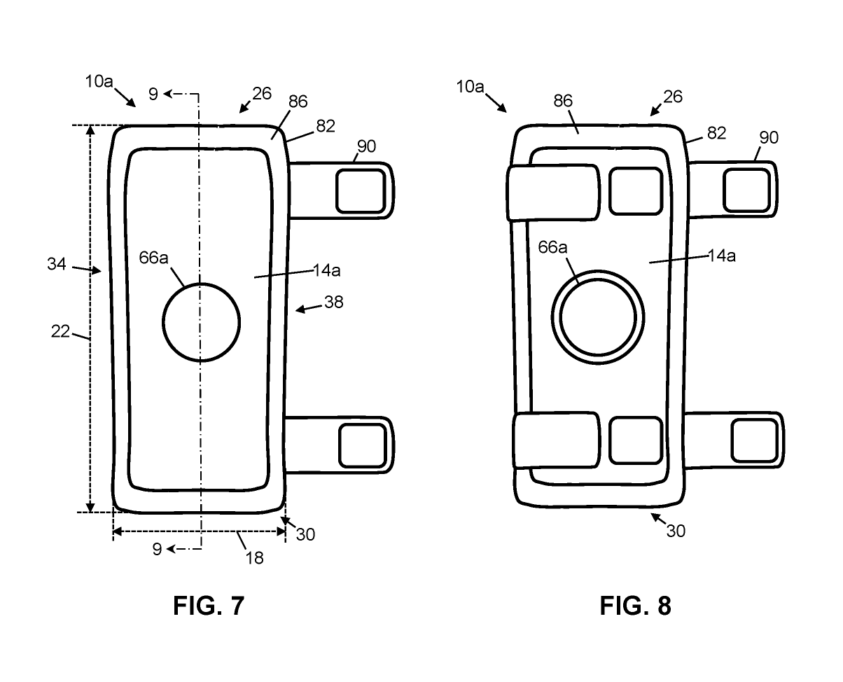

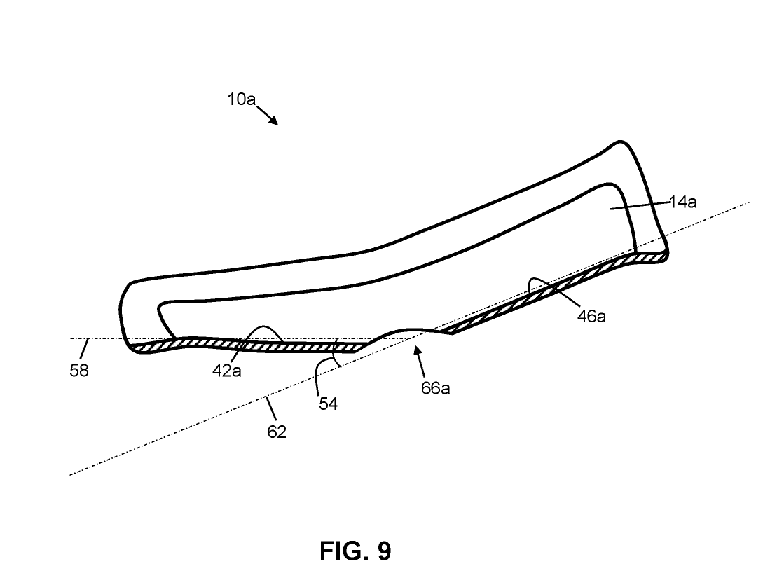

Referring now to FIGS. 6-10, shown therein and designated by the reference numeral 10a is a second embodiment of the present elbow braces. Brace 10a can be substantially similar to brace 10, with the primary exceptions described below. For example, in this embodiment, brace 10a includes a frame 14a having a first inner surface 42a and a second inner surface 46a, each of which may be configured to contact a posterior side 94 of arm 44.

In this embodiment, brace 10a includes an opening 66a extending through frame 14a and configured to support at least a portion of arm 44 (e.g., a portion of an elbow). As shown, opening 66a includes a circular shape. In other embodiments, an opening (e.g., 66a) may include a shape that is elliptical, otherwise rounded, rectangular, square, otherwise polygonal, and/or the like.

Similar to brace 10, brace 10a includes straps 90 configured to secure the brace to arm 44. In this embodiment, one or more straps 90 may extend over anterior side 50 of arm 44 such that intravenous (IV) therapy site 78 remains visible (e.g., between the straps) and such that a medical professional can visually assess, without removing the brace, whether the IV tubing and/or the catheter are in a proper position relative to the arm.

In this embodiment, brace 10a can be configured such that an IV tubing and/or a catheter can extend between straps 90 to access IV therapy site 78 on arm 44. Brace 10a may (e.g., also) be configured such that an IV tubing can extend between at least one strap 90 and arm 44 in order for a catheter to access IV therapy site 78 on the arm. At least by positioning the IV tubing between at least one strap 90 and arm 44, brace 10a prevents undesired disconnection of the IV tubing and/or catheter from the arm if the arm inadvertently and/or purposely moves.

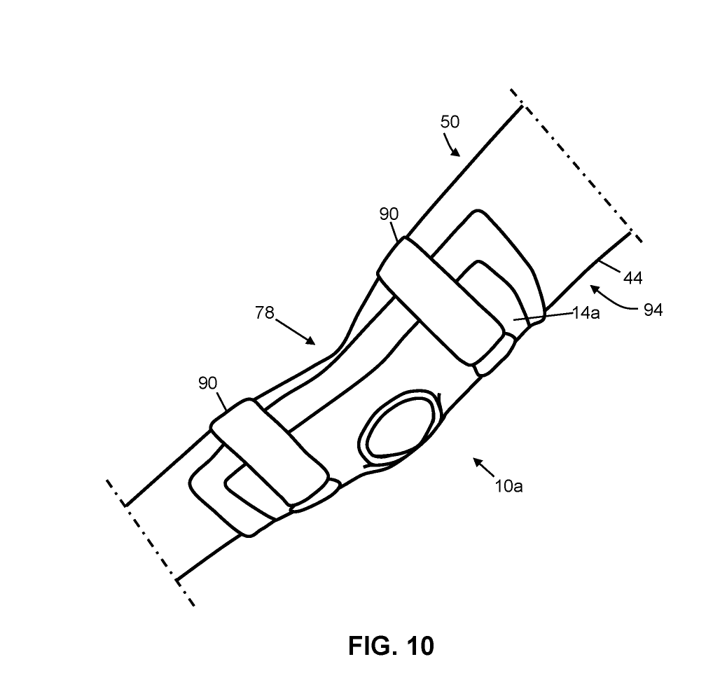

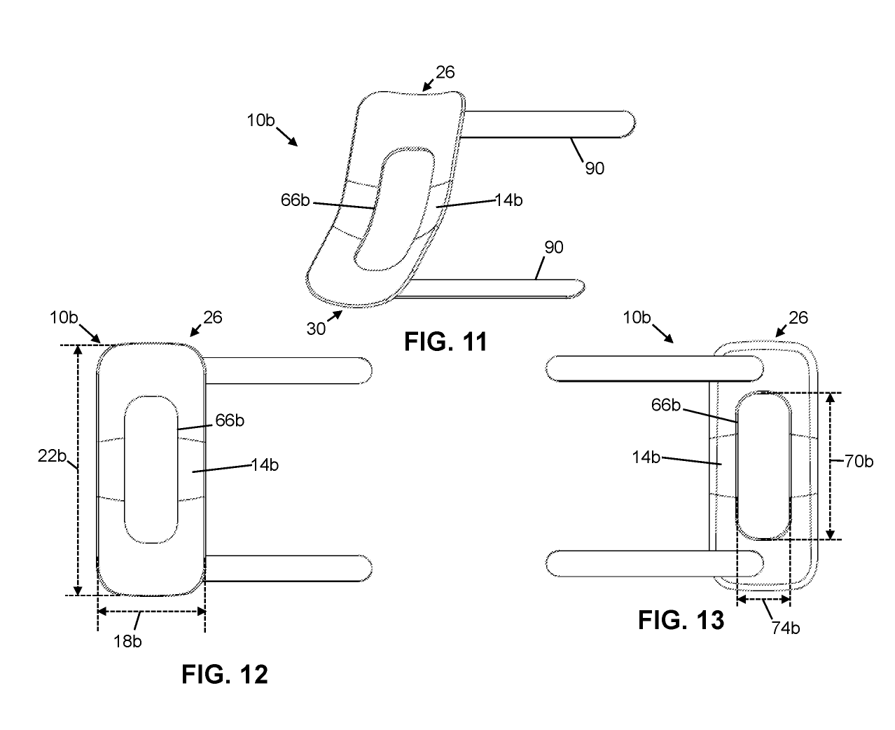

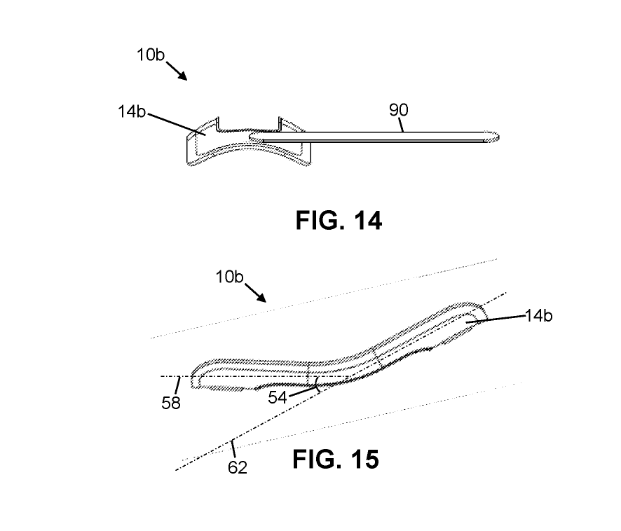

Referring now to FIGS. 11-15, shown therein and designated by the reference numeral 10b is a third embodiment of the present elbow braces. Brace 10b can be substantially similar to brace 10a, with the primary exceptions described below. For example, in this embodiment, brace 10b includes a frame 14b having an opening 66b extending through the frame. Opening 66b includes a length 70b and a width 74b, wherein the length is longer than the width. For example, length 70b of opening 66b may be at least approximately 50 percent (e.g., approximately any one of, or between any two of, the following: 50, 55, 60, 65, 70, 75, 80, 85, and 90 percent) of length 22b of frame 14b.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An elbow brace comprising:
a rigid frame, wherein the frame includes:
a width and a length longer than the width;
a first end having a first surface configured to contact an arm of a patient and a second end spaced along the length from the first end and having a second surface configured to contact the arm, wherein the second surface is configured to be disposed at an angle relative to the first surface that is between approximately 10 degrees and 45 degrees; and
an opening extending through the frame; and
a cushion configured to be disposed onto at least a portion of a peripheral edge of the frame.

2. The brace of claim 1, wherein:
the opening includes a length and a width, wherein the length is longer than the width; and
the length of the opening is at least half of the length of the frame.

3. The brace of claim 2, wherein the first surface and the second surface are configured to contact an anterior side of the arm.

4. The brace of claim 3, wherein the opening is sized such that, when the frame is disposed on the arm of the patient, an intravenous therapy site on the arm is visible through the opening.

5. The brace of claim 1, wherein the first surface and the second surface are configured to contact a posterior side of the arm.

6. The brace of claim 1, wherein the width has a substantially uniform maximum dimension between the first end and the second end of the frame.

7. The brace of claim 1, wherein the peripheral edge is raised relative to a portion of the frame such that, when the frame is disposed on the arm of the patient, the peripheral edge extends away from the arm.

8. An elbow brace comprising:
a rigid frame, wherein the frame includes:
a width and a length longer than the width, wherein the width has a substantially uniform maximum dimension between a first end of the frame and a second end of the frame;
an opening extending through the frame; and
a cushion covering configured to be disposed onto at least a portion of a peripheral edge of the frame.

9. The brace of claim 8, wherein the first end of the frame is spaced along the length of the frame from the second end of the frame, and wherein the first end includes a first surface configured to contact an arm of a patient and the second end includes a second surface configured to contact the arm, wherein the second surface is configured to be disposed at an angle relative to the first surface that is between approximately 10 degrees and 45 degrees.

10. The brace of claim 9, wherein the first surface and the second surface are configured to contact an anterior side of the patient's arm.

11. The brace of claim 9, wherein the opening is sized such that, when the frame is disposed on the arm of the patient, an intravenous therapy site is visible through the opening.

12. The brace of claim 9, wherein the first surface and the second surface are configured to contact a posterior side of the patient's arm.

13. The brace of claim 9, wherein the peripheral edge is raised relative to a portion of the frame such that, when the frame is disposed on the arm of the patient, the peripheral edge extends away from the patient's arm.

14. The brace of claim 1, comprising one or more straps, each of which comprise a first end and a second end, wherein, when the frame is disposed on the arm of the patient, at least one of the one or more support straps is configured to wrap around the arm of the patient such that the first end and the second end of the strap are coupled to the frame.

15. The brace of claim 1, wherein the frame comprises a thermoplastic material.
